(19) United States
(12) Patent Application Publication
(10) Pub. No.: US 2026/0193724 A1
(43) Pub. Date: Jul. 9, 2026

(54) METHOD FOR DETECTION OF SARS-COV-2 BY GOLD NANOPARTICLE ASSISTED LOOP MEDIATED ISOTHERMAL AMPLIFICATION (LAMP) AND KITS THEREOF

(71) Applicant: Imam Abdulrahman Bin Faisal University, Dammam (SA)

(72) Inventor: Huseyin TOMBULOGLU, Dammam (SA)

(73) Assignee: Imam Abdulrahman Bin Faisal University, Dammam (SA)

(21) Appl. No.: 19/013,700

(22) Filed: Jan. 8, 2025

(51) Int. Cl. C12Q 1/70; C12N 15/10; C12Q 1/6806; C12Q 1/6834; C12Q 1/6844

(52) U.S. Cl. CPC C12Q 1/701; C12N 15/1096; C12Q 1/6806; C12Q 1/6834; C12Q 1/6844

(57) ABSTRACT

A method for the detection of SARS-CoV-2 in a sample based on reverse transcription loop-mediated isothermal amplification (RT-LAMP) of a target nucleic acid sequence. The RT-LAMP amplicons are detected by gold nanoparticles (AuNPs) functionalized with a probe specific for regions of the target sequence. Kits for the detection of SARS-CoV-2 in a sample are also provided wherein the kit comprises a reverse transcriptase, a polymerase, a primer set for reverse transcription loop-mediated isothermal amplification (RT-LAMP) of the target sequence in a SARS-CoV nucleic acid sequence and variants thereof, and a conjugated nanoparticle solution comprising gold, water, and a probe sequence that is at least 85% identical to SEQ ID No.: 1.

### STATEMENT REGARDING PRIOR DISCLOSURE BY THE INVENTORS

Aspects of the present disclosure are described in Alhamid, G. et al., “Ultra-sensitive colorimetric detection of SARS-CoV-2 by novel gold nanoparticles (AuNP)-assisted loop-mediated isothermal amplification (LAMP) and freezing methods” published in Issue 6, Mikrochim Acta, which is incorporated herein by reference in its entirety.

### STATEMENT OF ACKNOWLEDGEMENT

Support provided by the Deanship of Scientific Research and the Institute for Research and Medical Consultations, Imam Abdulrahman Bin Faisal University, Saudi Arabia, through projects COVID19-2020-026-IRMC, 2020-IRMC-S-4, and 2020-IRMC-S-3 is gratefully acknowledged.

### REFERENCE TO SEQUENCE LISTING

In accordance with 37 CFR § 1.52 (e) (5) and with 37 CFR § 1.831, the specification makes reference to a Sequence Listing submitted electronically as a .xml file named “551723US Sequence Listing”. The .xml file was generated on Jan. 3, 2025, and is 7,009 bytes in size. The entire contents of the Sequence Listing are hereby incorporated by reference.

### BACKGROUND

### Technical Field

The present disclosure is directed to methods for the detection of SARS-CoV-2, and particularly, to methods based on loop-mediated isothermal amplification (LAMP) technique using gold nanoparticles (AuNP) functionalized with a probe complementary to the SARS-CoV-2 nucleic acid sequence.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Coronavirus was first identified during the outbreak of COVID-19 in the city of Wuhan, China. Severe acute respiratory syndrome coronavirus 2 (SARS-CoV-2) is a strain of coronavirus and is highly contagious. SARS-CoV-2 is transmitted from human to human via respiratory droplets from coughs and/or sneezes of an infected person. The rapid rate of multiplication of the virus led to a global pandemic and caused an alarming number of deaths. The severity of infection from SARS-CoV-2 may have lasting effects on the health of those infected. Since the virus has a high multiplication rate, its diagnosis and timely testing is important considering its deadly effects on the health of people.

Diagnosis of SARS-CoV-2 can be done using various methods including tests based on serology, antigen detection, and nucleic acid amplification. Molecular tests like nucleic acid amplification tests are preferred over other diagnostic methods because of their high sensitivity. Amongst these, polymerase chain reaction (PCR)-based assays are regarded as the gold standard for virus detection because of their high sensitivity and specificity [Shen, M. Z. et al., Recent advances and perspectives of nucleic acid detection for coronavirus, Journal of Pharmaceutical Analysis, 2020, 10, 97-101].

For detection of coronavirus, viral RNA is first transcribed to complimentary DNA (cDNA) by way of reverse transcription, which is followed by the amplification of cDNA by a polymerase chain reaction (PCR). Reverse transcription polymerase chain reaction (RT-PCR) is a laboratory technique that combines reverse transcription of RNA into DNA and amplification of specific DNA targets using polymerase chain reaction. The amplified nucleic acid is tagged with fluoresecent tags for detection of the nucleic acid [Adachi, D. et al., Comprehensive detection and identification of human coronaviruses, including the SARS-associated coronavirus, with a single RT-PCR assay, Journal of Virological Methods, 2004, 122, 29-36; and Guoguang, R. et al., COVID-19 Diagnostic Methods and Detection Techniques, Encyclopedia of Sensors and Biosensors, 2023, 17-32]. While RT-PCR determines the expression level of target genes, real time RT-PCR (also known as RT-qPCR) can be used to determine the copy number of the amplified target gene [Yiqun, M. et al., Application of Reverse Transcription-PCR and Real-Time PCR in Nanotoxicity Research, Methods Mol. Biol., 2012, 926, 99-112].

Recent advancements in diagnosis and testing methods based on PCR techniques include nucleic acid amplification at a constant temperature. Loop-mediated isothermal amplification (LAMP) is one such isothermal amplification technique wherein four to six primers bind to six to eight different regions of a target DNA at a fixed temperature. Amplification by LAMP can be completed in one step and may include reverse transcription that reduces detection time of the amplified nucleic acid. Although LAMP has advantages of isothermal reaction conditions and high sensitivity, LAMP reactions are still prone to mis-amplifications, leading to false-positive results. To overcome this problem, various colorimetric techniques and assays have been developed for a more accurate detection of amplicons. Fluorescent dyes, such as casein, pH sensitive dyes (such as cresol red and phenol red), and enzyme linked immunosorbent assays, have been developed for a sensitive and specific detection [Nupur, G. et al., Recent advances in loop-mediated isothermal amplification (LAMP) for rapid and efficient detection of pathogens, Curr. Res. Microb. Sci., 2022, 3, 100120].

Labeled gold nanoparticle(s) (AuNP(s)) probe assays offer sensitive, quick, simple, and low-cost testing alternatives to conventional diagnostic methods. Combination of LAMP and AuNP probes for colorimetric detection has been successfully developed for viral detection, including SARS-CoV-2. AuNPs in solution tend to form aggregates under different conditions and on addition of certain components. For example, AuNPs form aggregates upon salt induction, turning the solution color from blueish purple to colorless with time. With the presence of target DNA complementary to the hybridized probes, AuNP aggregation is prevented, and thus, the solution remains red. Probe-labeled AuNPs give rise to numerous applications in molecular diagnosis, gene editing, cancer labeling, and detection of pollutants [Sanromán-Iglesias, M. et al., The Role of Chemically Modified DNA in Discrimination of Single-Point Mutation through Plasmon-Based Colorimetric Assays, ACS Applied Nano Materials, 2018, 1, 7, 3741-3746; and Zhang, J. et al., Enhancing the stability of single-stranded DNA on gold nanoparticles as molecular machines through salt and acid regulation, Journal of Materials Chemistry B, 2019, 7, 5554-5562].

Prolonged incubation of AuNP-DNA conjugates leads to aggregation and may eventually lead to false negative results. There is a static repulsion between AuNPs-DNA conjugates due to the abundant negative charges in the phosphate backbone of DNA. This repulsion prevents AuNPs aggregation. The addition of salt in proper concentrations plays a role in the stabilization of the conjugates. Salt concentrations and type of salt have an impact on the stability of AuNP-DNA conjugates. For example, very high salt concentrations lead to irreversible aggregation and destabilization of AuNPs-DNA conjugates. Similarly, chloride and bromide salts stabilize adsorbed DNA more efficiently compared to other salts due to their higher affinity to AuNPs [Zhang, J. et al., Enhancing the stability of single-stranded DNA on gold nanoparticles as molecular machines through salt and acid regulation, Journal of Materials Chemistry B, 2019, 7, 5554-5562].

Various approaches have been made to improve detection methods using AuNP-DNA conjugates. AuNPs functionalized with N-gene probes have been developed to detect food-borne SARS-CoV-2. A drawback of the passive adsorption of probes to AuNPs is that certain conditions, like pH and salt concentration, can change with time and cause detachment and unstable structures, thus resulting in compromised assay sensitivity [Wang, J. et al., Gold nanoparticles in virus detection: Recent advances and potential considerations for SARS-CoV-2 testing developments, WIREs Nanomedicine and Nanobiotechnology, 2022, 14, 1, e1754.

Accordingly, an object of the present disclosure to provide a colorimetric detection method for SARS-CoV-2 using gold nanoparticles functionalized with single-stranded DNA probes which can provide faster and accurate results, and which can improve the efficiency of LAMP assays in point-of-care applications that may overcome drawbacks of the art.

### SUMMARY

In an exemplary embodiment, a method of detecting SARS-CoV-2 in a sample is described. The method includes contacting the sample with a primer set and one or more reverse transcription loop-mediated isothermal amplification (RT-LAMP) reagents to form a first reaction mixture, incubating the first reaction mixture at a first temperature and for a first time sufficient to amplify a target sequence of a SARS-CoV-2 nucleic acid sequence in the sample, mixing the incubated first reaction mixture with a conjugated nanoparticle solution to form a second reaction mixture, incubating the second reaction mixture at a second temperature and for a second time sufficient to hybridize the amplified target sequence of the SARS-CoV-2 nucleic acid sequence to the complex, assaying the sample with an assay to detect the hybridized target sequence of the SARS-CoV-2 nucleic acid sequence, and detecting the presence of the hybridized target sequence of the SARS-CoV-2 nucleic acid sequence, thereby detecting SARS-CoV-2 in the sample.

In some embodiments, the conjugated nanoparticle solution comprises gold, water, and a sequence that is at least 85% identical to SEQ ID No.: 1.

In some embodiments, the gold is in the form of nanoparticles.

In some embodiments, the SEQ ID No.: 1 is AAAAAAAAAAGCTGCAATATTGTTA ACGTG.

In some embodiments, the gold and the sequence that is at least 85% identical to SEQ ID No.: 1 form a complex.

In some embodiments, the sequence that is at least 85% identical to SEQ ID No.: 1 is a single stranded deoxyribonucleic acid sequence.

In some embodiments, at least 50% of the surface area of the gold is coated in the single stranded deoxyribonucleic acid sequence.

In some embodiments, the gold of the complex has a circumferential coating of the single stranded deoxyribonucleic acid sequence, wherein the single stranded deoxyribonucleic acid sequence has a longest dimension that is 0.1 to 10 nm.

In some embodiments, the primer set is E-ID1.

In some embodiments, a volumetric ratio of the first reaction mixture to the conjugated nanoparticles is from 1:5 to 5:1 based on a total volume of the first reaction mixture and the conjugated nanoparticles.

In some embodiments, the complex is in the form of spherical nanoparticles with an average diameter of 40 to 100 nm.

In some embodiments, the complex has a zeta potential of −10 to −20 mV.

In some embodiments, the complex has an ultraviolet-visible (UV-Vis) adsorption spectrum signal at 527 to 533 nm.

In some embodiments, a limit of detection of the assay is 0.5 to 0.6 fg/μL.

In some embodiments, the method includes incubating the first reaction mixture for 35 to 65 minutes.

In some embodiments, the method includes incubating the first reaction mixture at 50 to 80° C.

In some embodiments, the primer set comprises five primers, the primers having at least sequences of forward outer (F3), forward inner (FIP), backward outer (B3), backward inner (BIP), and loop backward (LB).

In some embodiments, the RT-LAMP reagents comprise at least a buffer, a magnesium salt, deoxyribonucleic triphosphates (dNTPs), at least one deoxyribose nucleic acid (DNA) polymerase, at least one reverse transcriptase, a template, and water.

In some embodiments, the conjugated nanoparticle solution is made by a process including mixing the sequence that is at least 85% identical to SEQ ID No.: 1 and gold nanoparticles in water to form a first mixture, incubating the first mixture at −100 to −60° C. for 30 to 90 minutes, thawing the first mixture to 2 to 6° C., washing the first mixture with a first buffer comprising a diethylpyrocarbonate-treated phosphate buffer and a salt to form a second mixture, centrifuging the second mixture, decanting a supernatant to leave a product, suspending the product in a second buffer comprising a diethylpyrocarbonate-treated phosphate buffer and a salt to form the conjugated nanoparticle solution.

In some embodiments, the method includes incubating the second reaction mixture for 0.5 to 10 minutes.

In some embodiments, the method includes incubating the second reaction mixture at 50 to 80° C.

In some embodiments, the assay is a colorimetric assay.

In some embodiments, the assay comprises mixing a salt solution with the hybridized target sequence of the SARS-CoV-2 nucleic acid sequence to detect the hybridized target sequence of the SARS-CoV-2 nucleic acid sequence.

In some embodiments, a volumetric ratio of the amplified target sequence of the SARS-CoV-2 nucleic acid sequence to the complex in the second reaction mixture is from 10:1 to 1:10.

In some embodiments, the salt solution has a concentration of 10 to 600 mM.

In some embodiments, the method comprises extracting nucleic acid from the sample.

In some embodiments, the method comprises treating a subject for which the sample was obtained.

In some embodiments, a kit for detection of SARS-COV in a sample is disclosed. The kit includes a reverse transcriptase, a polymerase, a primer set for reverse transcription loop-mediated isothermal amplification (RT-LAMP) of the target sequence in a SARS-COV nucleic acid sequence and variants thereof, and a conjugated nanoparticle solution comprising gold, water, and a sequence that is at least 85% identical to SEQ ID No.: 1. The primer set suitable for RT-LAMP, hybridizes to the target sequence of a SARS-COV nucleic acid sequence, and is configured to provide a positive result for the target sequence of a SARS-COV nucleic acid sequence in a predetermined assay time period otherwise determined for a positive sample of a target nucleic acid having a known sequence.

In some embodiments, any of the reagents in the kit may be combined in a mixture in a single container or provided in separate containers.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1 illustrates a flowchart depicting a method for detecting SARS-CoV-2 in a sample, according to certain embodiments.

FIG. 2 illustrates a systematic representation of the loop-mediated isothermal amplification (LAMP) conjugated AuNP-ssDNA hybrid and colorimetric detection of the SARS-CoV-2 E gene amplicons, according to certain embodiments.

FIG. 3A is a graph depicting zeta potential and a transmission electron microscope (TEM) image depicting the morphology of gold nanoparticles (AuNPs), according to certain embodiments.

FIG. 3B is a graph depicting zeta potential and a TEM image showing the morphology of AuNPs hybridized with an E gene probe, according to certain embodiments.

FIG. 3C depicts zeta potential (mV) of AuNPs and AuNPs hybridized with an E gene probe, according to certain embodiments.

FIG. 3D depicts the average size (nm) of AuNPs and AuNPs hybridized with an E gene probe, according to certain embodiments.

FIG. 3E depicts a UV-Vis absorption spectrum (350-700 nm) of AuNPs, according to certain embodiments.

FIG. 3F depicts a UV-Vis absorption spectrum (350-700 nm) of a dilution series of AuNPs hybridized with an E gene probe conjugate (1-107), according to certain embodiments.

FIG. 4A depicts visualization of positive and negative RT-LAMP reactions after the addition of AuNP-E probe and salt, where the accumulation of AuNPs is induced with the absence of viral amplicons, according to certain embodiments.

FIG. 4B depicts the effect of varying AuNP-E probe and LAMP amplicon ratios (8:2, 6:4, 4:6, and 2:8) on color development, according to certain embodiments.

FIG. 4C depicts the effect of varying salt concentrations (500, 250, 100, 50, 25, and 12.5 mM) on color development, according to certain embodiments.

FIG. 5A depicts results of gold nanoparticle-LAMP assays at different RT-LAMP reaction durations, according to certain embodiments.

FIG. 5B depicts results of color detection by a pH-dependent dye for a duration of up to 40 minutes, according to certain embodiments.

FIG. 5C depicts observations after loading post-RT-LAMP reactions in 2% agarose gel, according to certain embodiments.

FIG. 5D depicts serial dilutions of RT-LAMP amplicons (1-108), according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Wherever possible, corresponding, or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “nucleic acid” refers to biomolecules composed of nucleotides. A nucleic acid includes both deoxyribonucleic acid (DNA) and ribonucleic acid (RNA), including DNA and RNA containing non-standard nucleotides. A “nucleic acid” contains at least one polynucleotide (a “nucleic acid strand”). The term “nucleic acid”, as used herein, refers to either deoxyribonucleic acid (DNA), ribonucleic acid (RNA), single-stranded or double-stranded, and any chemical modifications thereof. The nucleic acid detected according to the systems, assays, and/or methods disclosed herein may be a full-length nucleic acid or a fragment thereof. Nucleic acids are biopolymers and macromolecules comprising nucleotides. A “nucleic acid” may be single-stranded or double-stranded. The term “nucleic acid” refers to nucleotides and nucleosides which make up, for example, DNA macromolecules and RNA macromolecules. The most common nucleic acids are DNA and RNA. In some embodiments, the target sequence is DNA or RNA.

As used herein, the term “nucleotide” refers to monomeric units of nucleic acids, DNA and RNA which are composed of a nitrogen containing base, pentose sugar and a phosphate. Nucleotides are monomer components comprising a five-carbon sugar, a phosphate group, and a nitrogenous base. The nitrogenous base, or nucleobase, may be adenine “A”, cytosine “C”, guanine “G”, thymine “T”, uracil “U”, and any nitrogenous base known in the art.

As used herein, the term “immunogenic” refers to the ability of a foreign substance (such as an antigen) to generate an immune response in the body of a human or an animal.

As used herein, the term “buffer” refers to a solution whose pH remains unaltered after dilution and/or adding small quantities of an acid or base at constant temperature.

As used herein, the term “nanoparticles” refers to particles whose size ranges between 1 to 100 nm. The term “nanoparticles” may also refer to particles having a size up to 500 nm.

As used herein, the term “particle size” may be considered as the longest lengths and/or dimensions of a particle.

As used herein, the term “primer” refers to a short single-stranded nucleotide sequence or oligonucleotide sequence complementary to a nucleic acid strand to be replicated. It may serve as a starting point for synthesizing a primer extension product. It may act as a point of initiation of synthesis when placed under conditions in which synthesis of primer extension product, which is complementary to a nucleic acid strand (template), is induced and/or in the presences of nucleotides and an agent for polymerization, such as DNA polymerase, and at a suitable temperature and pH. As used herein, the term “primer(s)” denotes a nucleic acid molecule that comprises at least 10 nucleotide residues and not more than 500 nucleotide residues, more preferably, not more than 200 nucleotide residues, still more preferably, not more than 100 nucleotide residues, and still more preferably, not more than 50 nucleotide residues, and that is capable of specifically hybridizing to the target sequence.

As used herein, the term “oligonucleotide(s)” refers to short DNA and/or RNA molecules, oligomers or oligos, used in a range of genetic testing and research. Oligonucleotides may be synthesized in a laboratory by solid-phase chemical synthesis and may be single-stranded molecules with any user-specified nucleic acid sequence. Oligonucleotides may be used for artificial gene synthesis, polymerase chain reaction (PCR), DNA sequencing, molecular cloning, as molecular probes, and the like. Oligonucleotides bind in a sequence-specific manner, to their respective complementary oligonucleotides, DNA, or RNA to form duplexes or, less often, hybrids of a higher order. Examples of procedures that use oligonucleotides include, but are not limited to, DNA microarrays, Southern blots, fluorescent in situ hybridization (FISH), PCR, the synthesis of artificial genes, and any procedure known in the art.

As used herein, the term “probe” refers to a single-stranded nucleic acid sequence that helps identify a specific nucleic acid sequence. A probe is designed to complement the nucleic acid sequence to be identified and is tagged with labels for easier detection. Probes have a high affinity and binding selectivity for one nucleic acid sequence and a high efficacy.

As used herein, the term “complementary” may be used in the context of a nucleic acid sequence capable of hybridizing with the target nucleic acid sequence based on complementary base pairing, i.e., adenine (A) of one sequence pairs with thymine (T) of another sequence, and cytosine (C) of one sequence always pairs with guanine (G) of another sequence. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases capable of hybridizing to at least one nucleic acid strand even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single stranded nucleic acid molecule.

As used herein, the term “conjugated nanoparticles” refers to nanoparticles attached to a probe sequence by intermolecular bonds, such as covalent and non-covalent bonds.

As used herein, the term “hybridization” refers to the binding of one nucleic acid sequence to another along its length to form a double-stranded sequence, wherein the binding is based on complementary base pairing between the sequences. As used herein, the term “hybridize” refers to a process of formation of double stranded nucleic acid regions between one, two, or many single stranded nucleic acid molecules complementary to one, two, or many target nucleic acid sequences. Hybridization may occur through specific hydrogen bonds between standard (Watson-Crick) base pair(s). Nucleic acid(s) that are “complementary” or “complement(s)” are those that are capable of base-pairing according to the standard Watson-Crick, Hoogsteen, or reverse Hoogsteen binding complementarity rules. As used herein, the term “specifically hybridizing” denotes the capability of a nucleic acid molecule to detectably anneal to another nucleic acid molecule, preferably a complementary nucleic acid molecule, under conditions in which such nucleic acid molecule does not detectably anneal to a non-complementary nucleic acid molecule.

As used herein, the term “amplification” refers to the synthesis of multiple copies of a nucleic acid sequence mediated by enzymes responsible for synthesis, such as polymerases. As used herein, the term “amplifying” or “amplification” denotes a process by which the number of copies of a gene is increased without a proportional increase in other genes.

As used herein, the term “amplicon” refers to the product of a nucleic acid sequence amplification.

A “target sequence,” as used herein, refers to a nucleic acid sequence of the SARS-CoV genome, or a compliment thereof, that is amplified, detected, or both amplified and detected using one or more oligonucleotides herein provided. Additionally, while the term target sequence sometimes refers to a double stranded nucleic acid sequence, those skilled in the art will also recognize that the target sequence can also be single stranded, e.g., RNA, as is the case herein. In an embodiment, the target sequence may be a “target nucleic acid” which, as used herein, refers to any nucleic acid that is targeted by primers to determine its presence in a sample. A target sequence may be selected that is more or less specific to an entire genus, to more than one genus, to a species or subspecies, serogroup, auxotype, serotype, strain, isolate or other subset of organisms. In some embodiments, the sample includes a body fluid such as blood, saliva, sweat, urine, lymph, feces, etc., or a nasal swab, an oral swab obtained from a subject. In some examples, the subject is an animal. In some examples, the subject is a mammal. In one example, the mammal is a human. In some embodiments, the sample is a purified nucleic acid, a nucleic acid in a biological fluid, or a nucleic acid preparation in the swab or body fluid. As used herein, the term “target nucleic acid” refers to a nucleic acid sequence, such as DNA or RNA, that is to be identified or detected by an assay.

As used herein, the term “colorimetric detection” refers to the discovery of a substance or a molecule in a solution based on the change in color of the solution.

Aspects of the present disclosure are directed to a method for detecting SARS-CoV-2 using loop-mediated isothermal amplification (LAMP). More particularly, the present disclosure relates to a gold nanoparticle (AuNP)-assisted colorimetric LAMP technique for diagnosing SARS-CoV-2, which aims to overcome false-positive results. In the present disclosure, the AuNPs are functionalized with E gene probes and introduced to LAMP amplicons using a freezing method. Varied salt concentrations and AuNP/E gene probe combinations were tested for the highest visual performance. The assay demonstrated good sensitivity, and the reaction results were achieved in 30 minutes. AuNP-assisted LAMP detection does not identify unspecific amplification, resulting in an efficient and accurate LAMP assay.

The disclosure provides a diagnostic method for a faster and more cost-effective detection of SARS-CoV-2 in samples from suspected patients. The detection time of 30 minutes may help to promptly treat the subject if infected. The test is performed at a fixed temperature, which helps to overcome the need for setting up sophisticated instruments and/or devices to run various thermal cycles, as in the case of PCR.

A subject may be tested for the presence of SARS-CoV-2 by taking a sample, such as a clinical sample, from the subject wherein the sample may contain nucleic acid of the suspected strain of the virus. The nucleic acid may be DNA or RNA. Such detection may be accomplished in situ or in vitro but is preferably conducted in vitro. As used herein, “sample” refers to any material or mixture of materials containing one or more analytes or entities of interest. As used herein, “analyte” refers to a substance that is suitable for testing in the present disclosure. The sample from the subject may be obtained from the saliva, sputum, serum, urine or blood of the subject. The sample may be obtained by invasive or non-invasive procedure depending on the type of sample to be taken. For example, the sample may be obtained non-invasively from the saliva of the subject or swabs taken from nasal, nasopharyngeal, and oropharyngeal areas. The clinical samples that may be evaluated in accordance with the present disclosure include any that may contain SARS-CoV-2, and include blood samples, bronchoalveolar lavage fluid specimens, fecal samples, fibrobronchoscope brush biopsy samples, nasal swab samples, nasopharyngeal swab samples, pharyngeal swab samples, oral samples (including saliva samples, sputum samples, and the like) and urine samples. Preferably, however, the employed clinical sample will be a nasal swab sample, a nasopharyngeal swab sample, a pharyngeal swab sample, or a sputum sample, and most preferably, the employed clinical sample will be a nasopharyngeal swab sample. In an embodiment, the sample may be pretreated to extract RNA that may be present in the sample. Alternatively, and more preferably, the sample will be evaluated without prior RNA extraction.

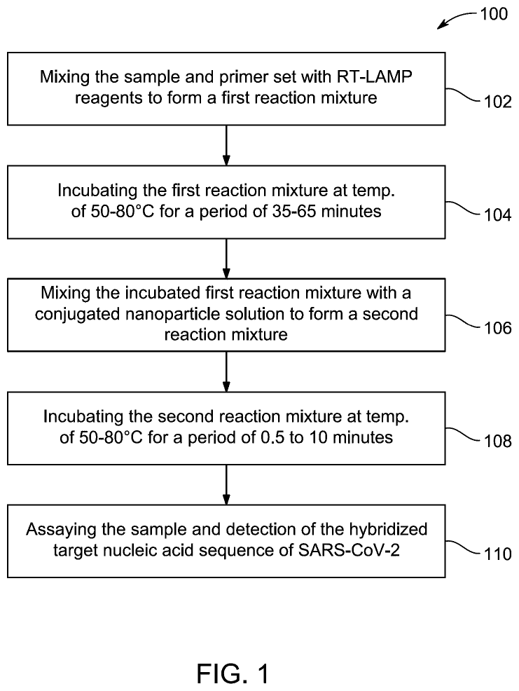

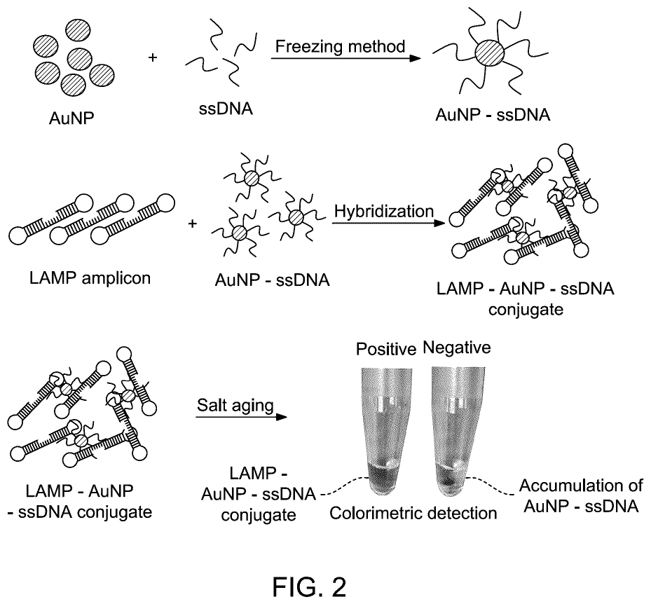

In an embodiment, a method 100 for detection of SARS-CoV-2 in a sample is described. A schematic flow diagram of the method 100 is shown in FIG. 1, and the method 100 is further illustrated with reference to the process depicted in FIG. 2. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes contacting the sample with one or more primers specific for the SARS-CoV-2 DNA. The primers are selected based on their specificity for regions of the target DNA. Typically, loop-mediated isothermal amplification (LAMP) uses a set of four to six primers, which include inner primers, outer primers, and loop primers. LAMP is a nucleic acid amplification method that relies on auto-cycle strand-displacement DNA synthesis performed by Bst DNA polymerase, or other strand displacement polymerases. In LAMP, four primers recognize six unique target sequences on a template strand. Two of the primers are designated as “inner primers” (FIP (forward inner primer) and BIP (backward inner primer)) and two are designated as “outer primers” (F3/FOP (forward outer primer) and B3/BOP (backward outer primer)). The reaction is initiated by annealing and extension of a pair of loop-forming primers (FIP and BIP), followed by annealing and extension of a pair of flanking primers (F3 and B3). Extension of these primers results in strand-displacement of loop-forming elements, which fold up to form hairpin-loop structures. In addition to containing a sequence that is complementary to a target sequence at their 3′ ends, the inner primers also contain a tail that comprises a sequence that is downstream of the 3′ end of primers in the template. Thus, an extension of an inner primer results in a product that has a self-complementary sequence at the 5′ end. Displacement of this product by an outer primer generates a product that has a loop at the 5′ end. Thus, the primer sets used in LAMP typically contain four, five, or six template-complementary sequences, where four sequences are found at the 3′ ends of the primers and two of the sequences are found at the 5′ ends of the primers. The initial reaction in LAMP results in a DNA product that has a dumbbell-like structure. In this product, the ends form stem loops and the single stranded region in between the stem loops is copied from the template. This product self-primes its own amplification to amplify the template sequence. LAMP uses a strand-displacing polymerase, and is isothermal, that is, it does not require heating and cooling cycles. The amplification process proceeds in an exponential manner (rather than a cyclic manner, like PCR) until all the nucleotides (dATP, dTTP, dCTP dGTP, dUTP, and the like) in the reaction mixture have been incorporated into the amplified DNA. Optionally, an additional pair of primers may be included to accelerate the reaction. These primers, termed loop primers (LF (loop forward primer) and LB (loop backward primer)), hybridize to non-inner primer bound terminal loops of the inner primer dumbbell shaped products.

Applications for LAMP have been further extended to include detection of RNA molecules by addition of reverse transcriptase (RT) enzyme. By including RNA detection, the types of targets for which LAMP can be applied are also expanded and add the ability to additionally target RNA based viruses, regulatory non-coding RNA (sRNA, miRNA), and RNA molecules that have been associated with particular disease or physiological states. The ability to detect RNA also has the potential to increase assay sensitivity, for instance, in choosing a highly expressed, stable, and/or abundant messenger RNA (mRNA) or ribosomal RNA (rRNA) targets. The preliminary phase of amplification involves the reverse transcription of RNA molecules to complementary DNA (cDNA). The cDNA then serves as template for the strand displacing DNA polymerase. Use of a thermostable RT enzyme enables the reaction to be completed at a single temperature and in a one step, single mix reaction.

In some embodiments, the sample is contacted with a set of at least 5 primers selected from a group consisting of a forward inner primer (FIP), a backward inner primer (BIP), a forward outer primer (FOP/F3), a backward outer primer (BOP/B3), a loop forward primer (LF), and a loop backward primer (LB). In a specific embodiment, the sample is contacted with a set of at least 5 primers consisting of a forward inner primer (FIP), a backward inner primer (BIP), a forward outer primer (FOP/F3), a backward outer primer (BOP/B3), and a loop backward primer (LB). The primer set may be selected based on the functional genes of SARS-CoV-2. In an exemplary embodiment, the primer set may be selected for an E gene of SARS-CoV-2.

The step 102 further includes mixing the sample and primer set with RT-LAMP reagents to form a first reaction mixture. The sample may comprise a SARS-CoV-2 nucleic acid sequence that serves as a template for cDNA synthesis. The template nucleic acid sequence may include a naturally occurring sequence or variants of a naturally occurring sequence. The RT-LAMP reagents may include a buffer to optimize the reaction conditions for binding the primers to the template nucleic acid sequence and to accelerate the activity of the polymerases. In some embodiments, the buffer may be suitable for LAMP use. In certain embodiments, the buffer is an isothermal amplification buffer. In some specific embodiments, the isothermal amplification buffer may typically include 20 mM Tris-HCl, 10 mM (NH₄)₂SO₄, 50 mM KCl, 2 mM MgSO₄, and 0.1% Tween 20. The proportions of the ingredients in the buffer may be varied according to the reaction conditions.

In some embodiments, the reagents in the first reaction mixture include deoxyribonucleoside triphosphates (dNTPs) as building blocks for amplification and transcription of nucleic acid sequences. The dNTPs may comprise deoxyadenosine triphosphate (dATP), deoxycytidine triphosphate (dCTP), deoxyguanosine triphosphate (dGTP) and deoxythymidine triphosphate (dTTP). In certain embodiments, the dNTPs may also include deoxyuridine Triphosphate (dUTP).

In certain embodiments, the reagents include metal ions for the colorimetric detection of the amplicons. Metal ions may enhance the incorporation of dNTPs into the nucleic acid sequence. In certain embodiments, the metal ions may be provided in a salt form. For example, the metal ions may be chloride, bromide, phosphate, and/or sulfate salts. In some embodiments, magnesium ions may be present as a magnesium salt. In an exemplary embodiment, magnesium sulfate (MgSO₄) may be added to the reaction mixture as the source of magnesium ions.

In some embodiments, the first reaction mixture includes at least one DNA polymerase to synthesize and amplify a target nucleic acid sequence. In certain embodiments, the at least one DNA polymerase may be a polymerase having strand displacement activity. The DNA polymerase may be selected from Bst 2.0 DNA polymerase, Bst 3.0 DNA polymerase, and/or omniAmp polymerase. In some embodiments, the polymerase is Bst 3.0 DNA polymerase. In another embodiment, the polymerase is Bst 2.0 DNA polymerase. In some embodiments, the reaction mixture may comprise a mixture of Bst 3.0 DNA polymerase and Bst 2.0 DNA polymerase. In some embodiments, the DNA polymerase is a WarmStart DNA polymerase. WarmStart DNA polymerase may be chosen due to its controlled activities and its ability to get activated at relatively lower temperatures. In a preferred embodiment, the DNA polymerase is a WarmStart Bst 2.0 DNA polymerase.

In some embodiments, the first reaction mixture includes at least one reverse transcriptase to transcribe SARS-CoV-2 RNA into cDNA. In certain embodiments, the at least one reverse transcriptase may be a polymerase having strand displacement activity. In other embodiments, the at least one reverse transcriptase may be an RTx reverse transcriptase. In some preferred embodiments, the reverse transcriptase may be a WarmStart RTx reverse transcriptase.

In certain embodiments, the first reaction mixture comprises water for mixing of the reagents and the sample. The water may be distilled water, double-distilled water, deionized water, and any other sterile filtered water free of nucleases. In a specific embodiment, the water may be deionized water (dH₂O). The RT-LAMP reagents may be added in a single step or in a sequential manner.

At step 104, the method 100 includes incubating the first reaction mixture under conditions sufficient to amplify a target sequence of the SARS-CoV-2 nucleic acid sequence in the sample. In some embodiments, the first reaction mixture is incubated at a temperature of 50-80° C. The specified temperature for the reaction can be obtained by using a thermal cycler, a water bath, a heating block or by means of a temperature sensor fitted into a LAMP device. In certain embodiments, the first reaction mixture is incubated at a temperature of 52-78° C., preferably 54-76° C., preferably 56-74° C., preferably 58-72° C., preferably 60-70° C., more preferably 62-68° C., and yet more preferably 64-66° C. In a preferred embodiment, the first reaction mixture is incubated at about 65° C.

In some embodiments, the first reaction mixture is incubated for 35-65 minutes (min). In other embodiments, the first reaction mixture is incubated for 40-60 min. In some other embodiments, the first reaction mixture is incubated for 45-55 min.

At step 106, the method 100 includes mixing the first reaction mixture from step 104 with a conjugated nanoparticle solution to form a second reaction mixture. Nanoparticles are a preferred choice in the present method as they are cost-effective, possess variable optical properties, and can interact with biomolecules with a high sensitivity. In some embodiments, the nanoparticles are gold nanoparticles (AuNPs).

AuNPs exhibit a surface plasmon resonance phenomenon (SPR) wherein smaller nanoparticles absorb light in the blue-green region of the visible spectrum and reflect red light. The absorption and reflection of light depend on the size and shape of the nanoparticles. Therefore, the optical properties of the AuNPs can be modulated by varying the particle size and shape. In some embodiments, the AuNPs may have spherical, spheroidal, polyhedral, and/or rod-like shapes. In a preferred embodiment, the AuNPs are spherical in shape. In some embodiments, the AuNPs have a particle size of 5-30 nm, preferably 628 nm, preferably 7-26 nm, preferably 8-24 nm, preferably 9-22 nm, preferably 10-20 nm, preferably 11-18 nm, preferably 12-16 nm, more preferably 13-15 nm, and yet more preferably about 14 nm.

In some embodiments, the AuNPs are conjugated to a probe having a single-stranded DNA sequence. In certain embodiments, the probe has a sequence complementary to the functionally important regions of the SARS-CoV-2 nucleic acid sequence. For example, the probe may be designed for an E gene of SARS-CoV-2 encoding the envelope protein of the virus. In other embodiments, the probe has a sequence at least 85% identical to SEQ ID No.: 1. In a specific embodiment, the probe sequence is a E-ID1 probe sequence.

The conjugated nanoparticles (AuNP-probe) may be provided as a solution with gold nanoparticles, the probe sequence, and water. The water may be distilled, double distilled, deionized, or any other sterile filtered water free of nucleases. The first reaction mixture is combined with a conjugated nanoparticle solution to form a second reaction mixture. In certain embodiments, the first reaction mixture is combined with the conjugated nanoparticle solution in a volumetric ratio of 1:5 to 5:1, preferably 1:4 to 4:1, preferably 3:1 to 3:1, preferably 2:1 to 2:1, and preferably about 1:1 based on the total volume of the first reaction mixture and the conjugated nanoparticle solution.

In some embodiments, the gold nanoparticles and the probe sequence form a complex. In some embodiments, the gold nanoparticles in the complex are coated with the probe sequence, wherein the probe sequence is circumferentially coated onto the gold nanoparticles. In an embodiment, at least 50%, preferably at least 55%, preferably at least 60%, preferably at least 65%, preferably at least 70%, preferably at least 75%, preferably at least 80%, preferably at least 85, preferably at least 90%, preferably at 95%, preferably at least 96%, preferably 97%, preferably at least 98%, more preferably at least 99%, and yet more preferably at least 99.5% of the surface area of the gold nanoparticles in the complex is coated with the probe sequence. In specific embodiments, the gold nanoparticles in the complex are coated with a single-stranded DNA sequence wherein the single-stranded DNA sequence is at least 85%, preferably at least 90%, preferably at least 95%, preferably at least 96%, preferably at least 97%, more preferably at least 98%, and yet more preferably at least 99% identical to SEQ ID No.: 1. The single-stranded DNA sequence in the complex has a longest dimension of about 0.1 to 10 nm, preferably 0.5 to 9 nm, preferably 1 to 8 nm, preferably 1.5 to 7 nm, preferably 2 to 6 nm, preferably 2.5 to 5 nm, or preferably 3 to 4 nm.

In some embodiments, the complex of gold nanoparticles and the probe sequence is in the form of spherical nanoparticles with an average diameter of 40 to 100 nm, preferably 45 to 95 nm, preferably 50 to 90 nm, preferably 55 to 85 nm, preferably 60 to 80 nm, and preferably 65 to 75 nm.

The complex of gold nanoparticles and the probe have a higher stability and show less aggregation when compared to just gold nanoparticles. In some embodiments, the complex has a zeta potential of −10 to −20 mV, preferably −12 to −19 mV, preferably, −14 to −18 mV, and more preferably −15 to −17 mV.

Further, the AuNPs in the complex exhibit optical properties after being functionalized with the probe. In some embodiments, the complex has an ultraviolet-visible (UV-Vis) spectrum signal at 527 to 533 nm, preferably 528 to 532 nm, more preferably 529 to 531 nm, and yet more preferably about 530 nm.

At step 108, the method 100 includes incubating the second reaction mixture under conditions sufficient to hybridize the amplified SARS-CoV-2 sequence (amplicons) to the complex. In an embodiment, a volumetric ratio of the amplicons to the complex in the second reaction mixture is from 10:1 to 1:10, preferably 9:1 to 1:9, preferably 8:1 to 1:8, preferably 7:1 to 1:7, preferably 6:1 to 1:6, preferably 5:1 to 1:5, preferably 4:1 to 1:4, preferably 3:1 to 1:3, preferably 2:1 to 1:2, and preferably about 1:1. In a preferred embodiment, the volumetric ratio of the amplicons to the complex in the second reaction mixture is about 7:3.

In some embodiments, the second reaction mixture is incubated at temperatures of 50-80° C., preferably 52-78° C., preferably 54-76° C., preferably 56-74° C., preferably 58-72° C., preferably 60-70° C., preferably 61-68° C., more preferably 62-66° C., and yet more preferably about 63° C.

The incubation of the second reaction mixture may be carried out for a period of 0.5 to 10 minutes, preferably 1 to 9 minutes, preferably 2 to 8 minutes, preferably 3 to 7 minutes, more preferably 4 to 6 minutes, and yet more preferably for about 5 minutes.

At step 110, the method 100 includes assaying the sample and detecting the presence of the hybridized target sequence of SARS-CoV-2 nucleic acid sequence in the sample. In some embodiments, the hybridized target sequence of SARS-CoV-2 nucleic acid sequence in the sample may be detected by a colorimetric assay. The assay comprises adding a salt solution to the second reaction mixture and detecting a change in the color of the mixture. In some embodiments, the reaction mixture is initially red in color, and adding salt may cause aggregation of conjugated nanoparticles, resulting in the change of color from red to colorless. In some embodiments, the color of the reaction mixture remains red in the presence of DNA, even after the addition of salt. In some embodiments, a positive result signifying the presence of the target nucleic acid sequence is indicated by a red color, while a negative result is indicated by a colorless mixture suggesting the absence of the target nucleic acid sequence.

The salt concentration may influence the intensity of color and, hence, may be adjusted to achieve a noticeable color distinction between the positive and negative results. In some embodiments, the salt solution may be added at a concentration of 10 to 600 mM, preferably 50 to 550 mM, preferably 70 to 500 mM, preferably 100 to 450 mM, preferably 150 to 400 mM, preferably 170 to 350 mM, preferably 200 to 300 mM, preferably 220 to 280 mM, more preferably 240 to 260 mM, and yet more preferably about 250 mM.

The sensitivity of the detection method using the AuNP probe conjugates is determined by the limit of detection (LoD) of the assay. In some embodiments, the limit of detection of the assay is about 0.5 to 0.6 fg/μL, preferably 0.51 to 0.59 fg/μL, preferably 0.52 to 0.58 fg/μL, preferably 0.53 to 0.57 fg/μL, and preferably 0.54 to 0.56 fg/μL.

In some embodiments, a method for the preparation of the conjugated nanoparticle solution is disclosed. The AuNPs are functionalized with the probe sequence using a freezing method. In some embodiments, the probe sequence and the gold nanoparticles are mixed with water to form a first mixture. The first mixture is incubated at a temperature of −100 to −60° C., preferably −95 to −65° C., preferably −90 to −70° C., more preferably −85 to −75° C., and yet more preferably about −80° C. Functionalization of nanoparticles with nucleic acid is found to occur at a faster rate at freezing temperatures. Moreover, the process does not require additional reagents for the completion of the process [Liu, B. and Liu, J., Freezing Directed Construction of Bio/Nano Interfaces: Reagentless Conjugation, Denser Spherical Nucleic Acids, and Better Nanoflares, Journal of the American Chemical Society, 2017, 139, 28, 9471-9474, which is incorporated herein by reference in its entirety].

In certain embodiments, the first mixture is incubated for a period of 30 to 90 minutes, preferably 35 to 85 minutes, preferably 40 to 80 minutes, preferably 45 to 75 minutes, preferably 50 to 70 minutes, more preferably 55 to 65 minutes, and yet mote preferably for about 60 minutes to functionalize the AuNPs with the probe.

Incubation of the first mixture is followed by thawing of the mixture at temperatures of 2 to 6° C., preferably 3 to 5° C., and more preferably about 4° C. The first mixture is then washed with a buffer and a salt to form a second mixture. The buffer may be any suitable buffer free of nucleases and/or may be treated to remove nucleases that may be present in the buffer. For example, the buffer may be a phosphate buffer and may be treated with diethylpyrocarbonate (DEPC) to remove nucleases. A salt may be added to the buffer solution to make it isotonic. In an exemplary embodiment, sodium chloride (NaCl) is added to the DEPC-treated buffer solution.

The second mixture is centrifuged at about 10,000-15,000 revolutions per minute (rpm), preferably 11,000 to 14,000 rpm, more preferably 11,500 to 13,000 rpm, and yet more preferably about 12,000 rpm for 25 to 40 minutes, preferably 30 to 35 minutes. The temperature for centrifugation may be kept at 30 to 50° C., preferably 35 to 45° C., or preferably about 40° C. Centrifugation is followed by decanting the supernatant to obtain the nanoparticle probe complexes. In some embodiments, the complexes are washed with a DEPC-treated phosphate buffer and salt, such as sodium chloride, to obtain the conjugated nanoparticles (AuNP-probe).

In some embodiments, the method of detection of SARS-CoV-2 in accordance with the present disclosure further includes extracting the nucleic acid from the sample. The extraction may be performed using methods known to a person skilled in the art. It may be done in a single step or multiple steps. In some embodiments, commercially available kits may be used for extraction.

In some embodiments, a kit is provided for the detection of SARS-Co-V-2 in a sample comprising the primer sets specific for the target SARS-CoV-2 nucleic acid sequence and its variants, a reverse transcriptase, a polymerase, AuNP-probe conjugates, and RT-LAMP reagents as described herein. In certain embodiments, the kit is configured to provide positive or negative results in a time period that is predetermined for a sample actually containing the target nucleic acid sequence. In an exemplary embodiment, the kit may provide positive results if the primer set hybridizes with the target nucleic acid sequence in an assay for a sample with the target nucleic acid sequence.

In one embodiment, the kit reagents may be provided as a mixture in a single container or separately in multiple containers. When provided in multiple containers, the reagents may be added sequentially and then thoroughly mixed. The methods herein may further include treating the subject whose sample shows a positive result for the presence of SARS-CoV-2 nucleic acid. The treatment may include following standard protocols mentioned in the COVID guidelines and administering immunogenic compositions according to the prescribed procedure.

### EXAMPLES

The following examples demonstrate the process of an RT-LAMP-mediated method for detecting SARS-CoV-2 as described herein. The examples are provided solely for illustration and are not to be construed as limitations of the present disclosure, as many variations thereof are possible without departing from the spirit and scope of the present disclosure.

### Example 1: Primer/Probe Design and RT-LAMP Reaction

RT-LAMP reaction setup and the E gene primer design were based on previous work [Alhamid, G. et al., SARS-CoV-2 detection methods: A comprehensive review, Saudi Journal of Biological Sciences, 2022, 29, 11, 103465, which is incorporated herein by reference in its entirety]. The LAMP primer mixture is comprised of five primers (F3, B3, FIP, BIP, LB). The E-ID1 probe contains d(A)₁₀ spacers at the 5′ end, which is complementary to the F1c-B1c regions. The primer and probe were synthetically synthesized (Bio Basic Inc., Canada) (Table 1). Briefly, an RT-LAMP reaction consisted of 2.5 μL 10× isothermal amplification buffer, 1.5 μL 100 mM MgSO₄, 3.5 μL 10 mM dNTP solution, 8,000 U/mL Bst 2.0 WarmStart DNA polymerase, 0.5 μL WarmStart RTx reverse transcriptase (New England BioLabs), 2.5 μL 10×LAMP primer mixture, 2 μL template (or dH₂O for NTC), and dH₂O up to 25 μL. The TECHNE TC152 thermal cycler (Keison products, UK) reacted at 65° C. for 40-60 minutes.

TABLE 1
Primer and probe sequences

| Primer/probe | SEQ ID No. | Sequence (5′-3′) |
|---|---|---|
| E-F3 | 3 | TCATTCGTTTCGGAAGAGA |
| E-B3 | 4 | AGGAACTCTAGAAGAATTCAGAT |
| E-FIP | 5 | TGTAACTAGCAAGAATACCACGAAACAGGTACGTTA |
| | | ATAGTTAATAGCG |
| E-BIP | 6 | GCTTCGATTGTGTGCGTACTCGAGAGTAAACGTAAAA |
| | | AGAAGG |
| E-LB | 7 | GCTGCAATATTGTTAACGTGAGTC |
| E-ID1 Probe | 1 | AAAAAAAAAAGCTGCAATATTGTTAACGTG |
| E-ID1 Proberc | 2 | AAAAAAAAAACGACGTTATAACAATTGCAC |

F3: forward outer primer, B3: backward outer primer, FIP: forward inner primer, BIP: backward inner primer, and LB: loop backward primer. Primer sequences were retrieved from Alhamid, G. et al. [Alhamid, G. et al., SARS-CoV-2 detection methods: A comprehensive review, Saudi Journal of Biological Sciences, 2022, 29, 11, 103465, which is incorporated herein by reference in its entirety].

### Example 2: Preparation of E-Probe-Modified AuNPs

The AuNPs and single-stranded E probe were conjugated by using the freezing method [Liu, B. and Liu, J., Freezing Directed Construction of Bio/Nano Interfaces: Reagentless Conjugation, Denser Spherical Nucleic Acids, and Better Nanoflares, Journal of the American Chemical Society, 2017, 139, 28, 9471-9474, which is incorporated herein by reference in its entirety]. Accordingly, 5 μL of 100 μM E-ID1 probe was mixed with 100 μL of AuNPs dispersion (Au 99.99%, 14 nm, 100 ppm in H₂O; US research Nanomaterials Inc., USA). The mixture was incubated at −80° C. for one hour and then left at room temperature to warm up to 4° C. The prepared mixtures were washed with 200 μL buffer A solution (PB, 0.1 M NaCl, pH 7.4, DEPC-treated), centrifuged at 12,000 rpm for 30 minutes at 4° C., and supernatants were carefully removed without disrupting the precipitated AuNP-E probe complexes. The washing step was repeated three times, and the final products were resuspended in 100 μL of buffer B (PB, 0.3 M NaCl, pH 7.4, DEPC-treated) and then kept at 4° C. until use.

### Example 3: Characterization of Nanoparticles and AuNP-E Conjugates

Zeta potential and dynamic light scattering (DLS) measurements were carried out on both AuNPs and the hybridized AuNP-E probes at 25° C. using Zetasizer Nano ZSP (Malvern Panalytical, UK) with three parallel measurements for each. Transmission electron microscopy (TEM) was performed on a Morgagni 268 (FEI) electron microscope with a Mega View G2 camera operating at an acceleration voltage of 80 kV. Samples were sonicated for 10 minutes and left to air dry for 30 minutes before being placed onto a copper grid precoated with a thin carbon film for observation. Serial dilutions of RT-LAMP products hybridized with AuNP-E probe were subjected to UV-Vis spectral analysis by transferring the reaction mixtures into a 96-well cell culture plate (Thermofisher Scientific). The absorbance values were read at a visual spectrum (300-700 nm) using a plate reader (BioTek Synergy HTX microplate reader, Agilent).

### Example 4: Detection of RT-LAMP Amplicons with AuNP-E Probe Conjugate

Post-LAMP reaction products were added to the AuNP-E probe, mixed by pipet, and incubated for 5 minutes at 63° C. for hybridization to obtain a colorimetric detection of RT-LAMP amplicons. After that, 5 μL MgSO₄ was added to the hybridized DNA AuNP-E probe solution, and the tubes were briefly centrifuged and left at room temperature. The addition of salt causes AuNPs-E probe aggregation, which results in a color change from red to colorless, while the presence of DNA prevents aggregation. The color remains red in positive samples (FIG. 2). Different AuNP-E probe and LAMP amplicon ratios (from 8:2 to 2:8 μL) and different salt concentrations (12.5-500 mM) were tested to select the concentrations that show the most distinctive color difference between positive and negative reactions. The detection sensitivity was evaluated by serially diluting the RT-LAMP product with a predetermined amplicon concentration by NanoDrop 2000c (Thermo Sci.) and subjecting it to the AuNP-E probe method to determine the limit of detection (LoD). Positive or negative RT-LAMP reactions were validated by loading RT-LAMP products in 2% agarose gel prepared with VisualaNA (A) DNA Stain from Molequle-On (Auckland, New Zealand) and run in an electrophoresis unit (Analytik Jena) for 45 minutes operating at 100 V. The gel was then visualized under a UV-trans illuminator (ChemiDoc™ XRS+ System with Image Lab™ Software, Bio-Rad, USA). Successful amplifications in positive samples were visualized as ladder-type DNA bands.

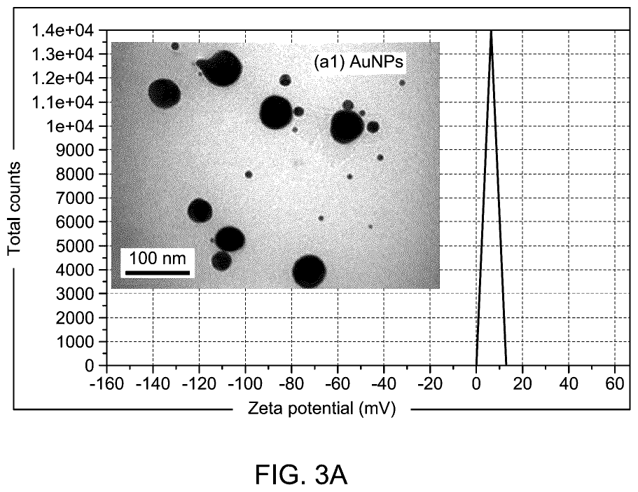

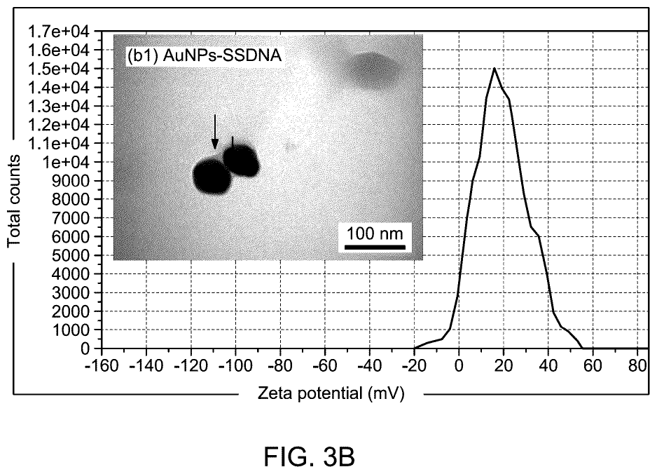

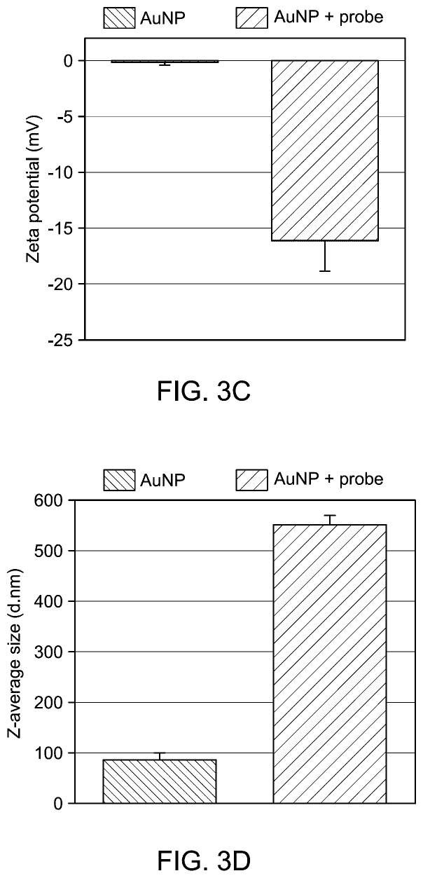

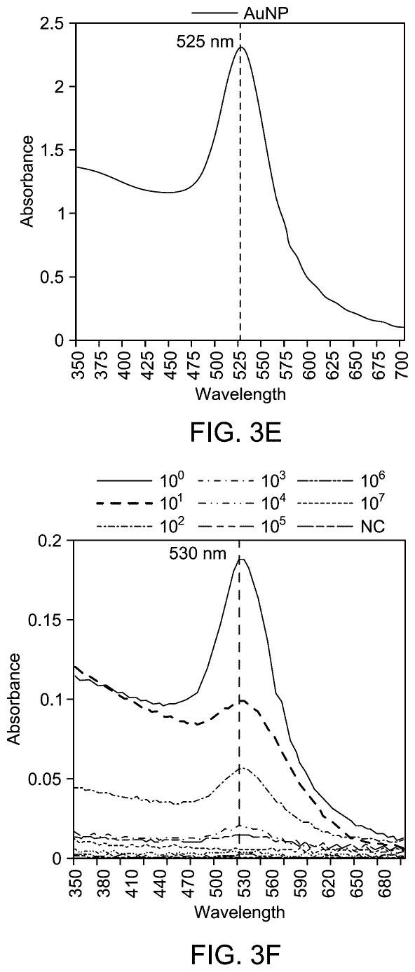

AuNPs and E probe-functionalized AuNPs were characterized using TEM to elucidate morphological properties. As seen in FIG. 3A, the TEM image of the AuNPs reveals a uniform and spherical morphology with an average size distribution of 14 nm. AuNPs demonstrate a homogeneous distribution and consistent interparticle spacing, contributing to the overall stability of the colloidal solution. TEM analysis of conjugation of the ssDNA (E probe) onto AuNPs is seen in FIG. 3B and demonstrates functionalization of AuNPs with E probes with no alteration of the spherical shape of the nanoparticles.

Zeta potential measurements were conducted to assess the surface charge of AuNPs. Upon functionalization with the E probe, there was an increase in the negative charge compared to bare AuNPs, as shown in FIG. 3C. The side-by-side comparison of zeta potentials for AuNPs and AuNP-E probe emphasizes the impact of functionalization on the overall charge of the nanoparticles. In addition, the DLS (zeta sizer) measurements demonstrated an increased hydrodynamic diameter after functionalizing AuNPs with the E probe (FIG. 3D).

Spectral analysis was conducted to investigate optical properties of the AuNPs and to determine functionalization of the E probe onto the AuNP surface. The UV-Vis absorption spectrum of bare AuNPs exhibits a prominent absorbance peak at 525 nm within the visual spectrum range (300-700 nm), with an absorbance value of 2.307 (FIG. 3E). This peak is characteristic of the surface plasmon resonance (SPR) of AuNPs and is indicative of their optical properties. Following functionalization with the probe and serial dilutions, the UV-Vis absorption spectrum reveals a shift in the absorbance peak to 530 nm, with reduced absorbance values (FIG. 3E).

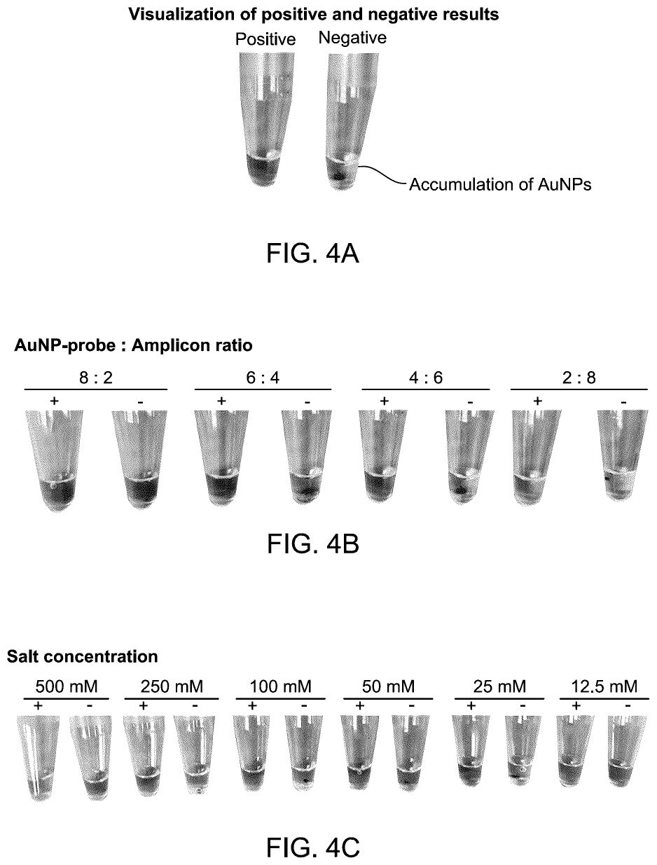

Visualization of positive and negative RT-LAMP results was first tested by adding the AuNP-E probe to post-RT-LAMP reaction tubes with no colorimetric or fluorometric indicator. Positive RT-LAMP amplicons remained red after adding salt. In negative RT-LAMP reactions or the absence of viral amplicons, AuNPs visibly aggregate upon salt addition, and the solution turns colorless (FIG. 4A).

The effect of AuNP-E probe concentration on hybridization with LAMP amplicons was tested with various probe ratios to LAMP amplicons. With the AuNP probe at a ratio of 8:2-2:8 with amplicons, followed by salt addition (final concentration, 30 mM MgSO₄), the ratios between 8:2 and 6:4 did not show a large color change between the original red (positive result) and purplish-white (negative result). In contrast, ratios between 4:6 and 2:8 gave a more prominent difference (FIG. 4B). A ratio between 4:6 and 2:8 was chosen because it showed a better color difference. Therefore, a 3:7 ratio was selected as the hybridization condition.

The effect of salt concentration on aggregation was tested by adding MgSO₄ at various concentrations (FIG. 4C). No color difference between positive and negative LAMP reactions was observed at the highest (500 mM) and lowest (12.5 mM) salt concentrations. Lower salt concentrations were avoided to avoid the interpretation of false positive results. A salt concentration of 250 mM was chosen for showing the most apparent color difference and the most proper aggregation in negative samples without leading to false negative results in the infected (positive) samples.

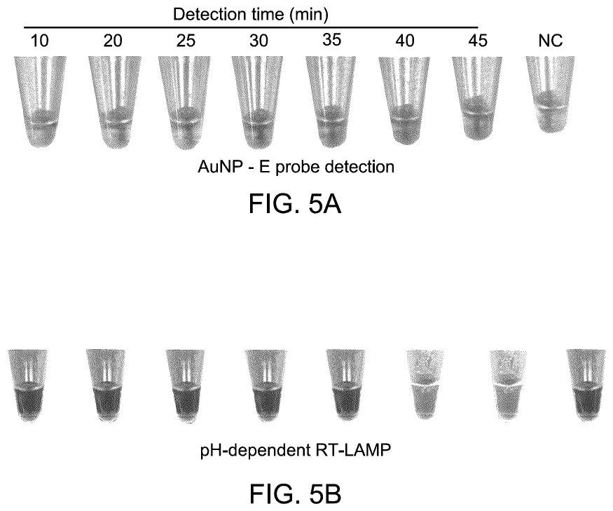

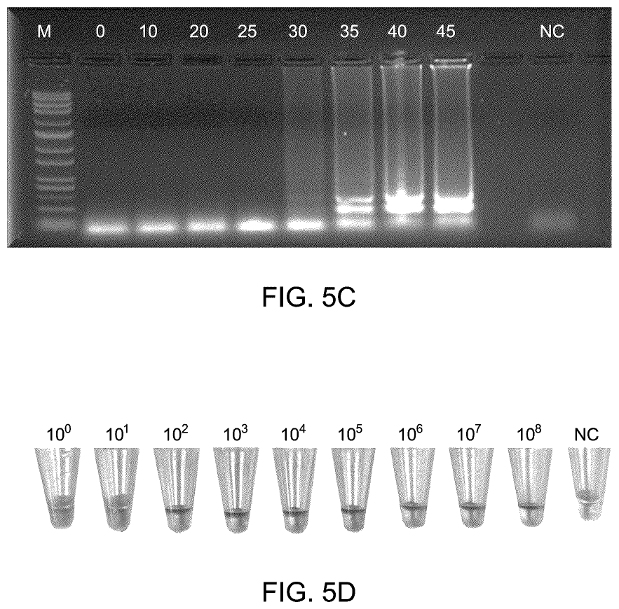

To determine a minimum RT-LAMP reaction time to observe a color difference, the ability of the AuNP-E probe to detect SARS-CoV-2 amplicons at various reaction times ranging from 10 to 45 minutes was evaluated (FIG. 5A). An RT-LAMP reaction with a colorimetric indicator was also run simultaneously for reference. The AuNP-E probe detected the SARS-CoV-2 amplicons after a 30-minute reaction time, which was faster than the pH-dependent color change in the reference reaction, as shown in FIG. 5B. This observation was corroborated by loading post-RT-LAMP reactions in a 2% agarose gel, supporting amplicon production at 30 minutes (FIG. 5C).

To assess the sensitivity of the AuNP-E probe, serial dilutions of RT-LAMP reaction products containing a known copy number of SARS-CoV-2 RNA (1 million copies/μL) were performed. The probe's ability to detect the virus at varying dilutions was evaluated. The AuNP-E probe demonstrated sensitivity in detecting SARS-CoV-2 RNA, as shown in FIG. 5D.

### Discussion

Labeled AuNPs probe assays represent a trend for the visual detection of pathogen as they offer sensitive, quick, simple, and low-cost testing alternatives to conventional diagnostic methods. Compared to citrate-stabilized AuNPs, AuNPs coated with DNA are less prone to salt-induced aggregation [Iglesias, M. S. and Grzelczak, M., Using gold nanoparticles to detect single-nucleotide polymorphisms: Toward liquid biopsy, Beilstein Journal of Nanotechnology, 2020, 11, 263-284, which is incorporated herein by reference in its entirety]. TEM images revealed a stable dispersion of AuNP-E probe complexes, indicating a well-dispersed and functionalized state (FIG. 3A and FIG. 3B). A colorimetric AuNP-probe assay detected SNP of breast cancer in patients' liquid biopsies and found that larger diameters promoted a higher sensitivity, while the specificity was higher when smaller particle diameters were used [Sanromán-Iglesias, M. et al., The Role of Chemically Modified DNA in Discrimination of Single Point Mutation through Plasmon-Based Colorimetric Assays, ACS Applied Nano Materials, 2018, 1, 7, 3741-3746, which is incorporated herein by reference in its entirety]. The DLS measurements showed different hydrodynamic parameters before and after the hybridization of probes to AuNPs, as shown in FIG. 3D, indicating a successful labeling process. The comparative analysis of size distribution profiles highlighted the impact of functionalization on the hydrodynamic diameter of the nanoparticles, providing insights into the stability and structure of the AuNP-E probe hybrid.

Appropriate RT-LAMP amplicon to AuNPs-probe ratios were determined for consistency and reliability. As shown in FIG. 4B, lower RT-LAMP amplicon and higher AuNPs-probe ratios lead to false positive results, while less AuNPs-probe and higher RT-LAMP amplicon ratios dilute the solution color and lead to indistinguishable visual results.

AuNPs exhibit SPR absorption characteristics in the visible light spectrum that depend on the interparticle distance and their aggregation results in a shift in the absorption band accompanied by a solution color change from red to purplish-blue to colorless. This property was utilized for solution-phase colorimetric detection of specific DNA sequences for molecular diagnosis applications. AuNPs give a peak at 525 nm in the UV-Vis spectrum, while those hybridized with probes exhibit a shift to 530 nm (5 nm shift) [Jaroenram, W. et al., Rapid and sensitive detection of shrimp yellow head virus using loop-mediated isothermal amplification and a colorogenic nanogold hybridization probe, Journal of Virological Methods, 2012, 186, 1-2, 36-42; and Ye, X. et al., Gold nanoparticle mediated nucleic acid isothermal amplification with enhanced specificity, Analytica Chimica Acta, 2018, 1043, 150-157, which are incorporated herein by references in their entireties]. This phenomenon was also observed in the present disclosure, indicating hybridization. The observed changes in the absorption spectrum indicate alterations in the local environment of the AuNPs due to the attachment of ssDNA, suggesting a change in the electronic structure of the nanoparticles.

Thiol-labeled DNA attachment to AuNPs is the most common approach; however, it is time-consuming and expensive [Zhang, J. et al., Enhancing the stability of single-stranded DNA on gold nanoparticles as molecular machines through salt and acid regulation, Journal of Materials Chemistry B, 2019, 7, 5554-5562, which is incorporated herein by reference in its entirety]. A freezing method of DNA attachment to AuNPs results in a higher DNA density, higher stability, and lower cost since no extra reagents are required to achieve hybridization [Liu, B. and Liu, J., Freezing Directed Construction of Bio/Nano Interfaces: Reagentless Conjugation, Denser Spherical Nucleic Acids, and Better Nanoflares, Journal of the American Chemical Society, 2017, 139, 28, 9471-9474, which is incorporated herein by reference in its entirety]. The current disclosure successfully detected the SARS-CoV-2 virus based on a color change of a solution containing AuNPs and probes hybridized to LAMP amplicons with a high sensitivity of 5.635 pg/μL, corresponding to 5.635 ng/ml. The functionalization of AuNPs was done based on poly(A) probe affinity to the gold surface simply via freezing AuNP solution with poly A-labeled E gene probes, without needing additional reagents or extended processing time. A colorimetric RT-LAMP assay gives the earliest positive reaction at a time of about 40 minutes (FIG. 5B). Once compared with this assay using RNA as the template, the current AuNP-LAMP assay leads a detection time of 30 minutes (FIG. 5A). This approach is cost-effective and time-effective and allows new opportunities in a wide range of diagnostic developments.

Numerous modifications and variations of the present disclosure are possible in light of the above teachings. It is therefore to be understood that within the scope of the appended claims, the disclosure may be practiced otherwise than as specifically described herein.

1: A method of detecting SARS-CoV-2 in a sample, comprising:
contacting the sample with a primer set and one or more reverse transcription loop-mediated isothermal amplification (RT-LAMP) reagents to form a first reaction mixture;
incubating the first reaction mixture at a first temperature and for a first time sufficient to amplify a target sequence of a SARS-CoV-2 nucleic acid sequence in the sample;
mixing the incubated first reaction mixture with a conjugated nanoparticle solution to form a second reaction mixture,
wherein the conjugated nanoparticle solution comprises gold, water, and a sequence that is at least 85% identical to SEQ ID No.: 1,
wherein the gold is in the form of nanoparticles,
wherein SEQ ID No.: 1 is AAAAAAAAAAGCTGCAATATTGTTAACGTG,
wherein the gold and the sequence that is at least 85% identical to SEQ ID No.: 1 form a complex,
wherein the sequence that is at least 85% identical to SEQ ID No.: 1 is a single stranded deoxyribonucleic acid sequence,
wherein at least 50% of a surface area of the gold is coated in the single stranded deoxyribonucleic acid sequence,
wherein the gold of the complex has a circumferential coating of the single stranded deoxyribonucleic acid sequence,
wherein the single stranded deoxyribonucleic acid sequence has a longest dimension that is 0.1 to 10 nm,
incubating the second reaction mixture at a second temperature and for a second time sufficient to hybridize the amplified target sequence of the SARS-CoV-2 nucleic acid sequence to the complex;
assaying the sample with an assay to detect the hybridized target sequence of the SARS-CoV-2 nucleic acid sequence; and
detecting a presence of the hybridized target sequence of the SARS-CoV-2 nucleic acid sequence, thereby detecting SARS-CoV-2 in the sample,
wherein the primer set is E-ID1.

2: The method of claim 1, wherein a volumetric ratio of the first reaction mixture to the conjugated nanoparticles is from 1:5 to 5:1 based on a total volume of the first reaction mixture and the conjugated nanoparticles.

3: The method of claim 1, wherein the complex is in the form of spherical nanoparticles with an average diameter of 40 to 100 nm.

4: The method of claim 1, wherein the complex has a zeta potential of −10 to −20 mV.

5: The method of claim 1, wherein the complex has an ultraviolet-visible (UV-Vis) adsorption spectrum signal at 527 to 533 nm.

6: The method of claim 1, wherein a limit of detection of the assay is 0.5 to 0.6 fg/μL.

7: The method of claim 1, wherein incubating the first reaction mixture occurs for 35 to 65 minutes.

8: The method of claim 1, wherein incubating the first reaction mixture occurs at 50 to 80° C.

9: The method of claim 1, wherein the primer set comprises five primers, the primers having at least sequences of forward outer (F3), forward inner (FIP), backward outer (B3), backward inner (BIP), and loop backward (LB).

10: The method of claim 1, wherein the RT-LAMP reagents comprise at least a buffer, a magnesium salt, deoxyribonucleic triphosphates (dNTPs), at least one deoxyribose nucleic acid (DNA) polymerase, at least one reverse transcriptase, a template, and water.

11: The method of claim 1, wherein conjugated nanoparticle solution is made by a process comprising:
mixing the sequence that is at least 85% identical to SEQ ID No.: 1 and gold nanoparticles in water to form a first mixture;
incubating the first mixture at −100 to −60° C. for 30 to 90 minutes;
thawing the first mixture to 2 to 6° C.;
washing the first mixture with a first buffer comprising a diethylpyrocarbonate-treated phosphate buffer and a salt to form a second mixture;
centrifuging the second mixture;
decanting a supernatant to leave a product;
suspending the product in a second buffer comprising a diethylpyrocarbonate-treated phosphate buffer and a salt to form the conjugated nanoparticle solution.

12: The method of claim 1, wherein incubating the second reaction mixture occurs for 0.5 to 10 minutes.

13: The method of claim 1, wherein incubating the second reaction mixture occurs at 50 to 80° C.

14: The method of claim 1, wherein the assay is a colorimetric assay.

15: The method of claim 1, wherein the assay comprises mixing a salt solution with the hybridized target sequence of the SARS-CoV-2 nucleic acid sequence to detect the hybridized target sequence of the SARS-CoV-2 nucleic acid sequence.

16: The method of claim 1, wherein a volumetric ratio of the amplified target sequence of the SARS-CoV-2 nucleic acid sequence to the complex in the second reaction mixture is from 10:1 to 1:10.

17: The method of claim 15, wherein the salt solution has a concentration of 10 to 600 mM.

18: The method of claim 1, further comprising:
extracting nucleic acid from the sample.

19: The method of claim 1, further comprising:
treating a subject for which the sample was obtained.

20: A kit for detection of SARS-COV in a sample, the kit comprising:
a reverse transcriptase, a polymerase, a primer set for reverse transcription loop-mediated isothermal amplification (RT-LAMP) of the target sequence in a SARS-COV nucleic acid sequence and variants thereof, and conjugated nanoparticle solution comprising gold, water, and a sequence that is at least 85% identical to SEQ ID No.: 1;
wherein the primer set suitable for RT-LAMP hybridizes to the target sequence of a SARS-CoV nucleic acid sequence is configured to provide a positive result for the target sequence of a SARS-COV nucleic acid sequence in a predetermined assay time period otherwise determined for a positive sample of a target nucleic acid having a known sequence;
wherein any of the reagents in the kit may be combined in a mixture in a single container or provided in separate containers.


SEQUENCE LISTING (7 sequences)

SEQ ID NO: 1 - RNA, 30 nt, synthetic construct
  source 1..30: mol_type = other RNA
aaaaaaaaaagctgcaatattgttaacgtg

SEQ ID NO: 2 - RNA, 30 nt, synthetic construct
  source 1..30: mol_type = other RNA
aaaaaaaaaacgacgttataacaattgcac

SEQ ID NO: 3 - RNA, 19 nt, synthetic construct
  source 1..19: mol_type = other RNA
tcattcgtttcggaagaga

SEQ ID NO: 4 - RNA, 23 nt, synthetic construct
  source 1..23: mol_type = other RNA
aggaactctagaagaattcagat

SEQ ID NO: 5 - RNA, 49 nt, synthetic construct
  source 1..49: mol_type = other RNA
tgtaactagcaagaataccacgaaacaggtacgttaatagttaatagcg

SEQ ID NO: 6 - RNA, 43 nt, synthetic construct
  source 1..43: mol_type = other RNA
gcttcgattgtgtgcgtactcgagagtaaacgtaaaaagaagg

SEQ ID NO: 7 - RNA, 24 nt, synthetic construct
  source 1..24: mol_type = other RNA
gctgcaatattgttaacgtgagtc